(19) United States
(12) Patent Application Publication
(10) Pub. No.: US 2024/0386583 A1
(43) Pub. Date: Nov. 21, 2024

(54) MEDICAL IMAGE PROCESSING APPARATUS, X-RAY DIAGNOSIS APPARATUS, AND MEDICAL IMAGE PROCESSING METHOD

(71) Applicant: CANON MEDICAL SYSTEMS CORPORATION, Otawara-shi (JP)

(72) Inventors: Yoshiteru KOBAYASHI, Utsunomiya (JP); Takuya AIDA, Nasushiobara (JP); Saki YOSHIDA, Shimoda (JP); Mika TAKAYA, Nasushiobara (JP); Hirona OIKAWA, Nasushiobara (JP); Yuki KOIKE, Otawara (JP)

(73) Assignee: CANON MEDICAL SYSTEMS CORPORATION, Otawara-shi (JP)

(21) Appl. No.: 18/654,438

(22) Filed: May 3, 2024

(30) Foreign Application Priority Data
May 18, 2023 (JP) 2023-082084

(51) Int. Cl. G06T 7/33; A61B 6/00; A61B 6/12; A61B 6/46; A61B 6/50; A61B 34/20; G06T 7/00; G06T 7/60; G06T 7/73

(52) U.S. Cl. CPC G06T 7/33; A61B 6/12; A61B 6/463; A61B 6/487; A61B 6/504; A61B 34/20; G06T 7/0012; G06T 7/60; G06T 7/73; G06T 2207/10088; G06T 2207/10121; G06T 2207/30016; G06T 2207/30101

(57) ABSTRACT

A medical image processing apparatus includes processing circuitry. The processing circuitry obtains activity position data related to positions of activity regions that bring a site of an examined subject into an activity and a blood vessel image rendering a three-dimensional blood vessel related to the activity regions. The processing circuitry performs a registration between the activity position data and the blood vessel image. The processing circuitry calculates a positional relationship between at least one of the activity regions and the blood vessel, on the basis of a result of the registration. On the basis of the positional relationship, the processing circuitry causes a display to display information about positioning a device capable of receiving, within the blood vessel of the examined subject, electrical signals from the activity regions.

### CROSS-REFERENCE TO RELATED APPLICATIONS

This application is based upon and claims the benefit of priority from Japanese Patent Application No. 2023-082084, filed on May 18, 2023, the entire contents of which are incorporated herein by reference.

### FIELD

Embodiments described herein relate generally to a medical image processing apparatus, an X-ray diagnosis apparatus, and a medical image processing method.

### BACKGROUND

Conventionally, for treating aortic aneurysms and myocardial infarctions, stents placed in blood vessels of patients are clinically used. An experiment is being developed by which, for example, a reception electrode is attached to one of such stents so as to obtain data of an electrical signal from the motor cortex of the brain. If using the stent having such an electrode (hereinafter, “electrode-bearing stent”) can make it possible to control a digital machine with information from a thinking process of the brain, there is a possibility that this technology may serve as a new option for patients with a motor neuron disorder. Human clinical tests of electrode-bearing stents are already underway, and there is a high possibility that this technology comes into practical use in the near future.

Placement of electrode-bearing stents is different from treatment using normal stents (stents having no electrode). In other words, in the placement location of an electrode-bearing stent, there is no aneurysm or stenosis of a blood vessel that can serve as a landmark of the placement location. Further, veins in which an electrode-bearing stent can be placed are extremely thin and can easily break, as compared to arteries. For these reasons, placement of an electrode-bearing stent is considered to have a higher degree of difficulty than placement of a stent in an artery, which is commonly practiced in intravascular treatment.

Further, conventionally, it is possible to recognize the position and to capture an image of a neural activity area of the brain by using functional Magnetic Resonance Imaging (fMRI). The fMRI technology makes it possible to recognize the neural activity area of the brain in a non-invasive manner and to also visualize which part of the brain has an activity while a goal task is performed. For this reason, also at a preparation stage before placing an electrode-bearing stent, a neural activity area of the brain is recognized by using fMRI, the vein for the placement is three-dimensionally (3D) imaged, and a goal position is marked.

A registration between the neural activity area of the brain and a blood vessel as well as the marking are manually performed by a user. For this reason, determining the placement position of the electrode-bearing stent is entrusted to experience of the practitioner. Further, the practitioner is supposed to perform manipulations by visually comparing an angiography fluoroscopic image and a marked model image that are displayed separately from each other.

Consequently, in order to accurately and safely place an electrode-bearing stent in an optimal placement location for the electrode-bearing stent, advanced skills and experience are required. In other words, difficult problems are posed by setting the optimal placement location for an electrode-bearing stent and accurately and safely placing the electrode-bearing stent in the optimal placement location for the electrode-bearing stent.

### BRIEF DESCRIPTION OF THE DRAWINGS

FIG. 1 is a block diagram illustrating an exemplary configuration of a medical information processing system 1 including a medical image processing apparatus according to an embodiment;

FIG. 2 is a drawing according to an embodiment illustrating an example of an activity blood vessel superimposed image generated through a registration between a blood vessel image and activity position data;

FIG. 3 is a drawing according to the embodiment illustrating examples of distances between a plurality of activity regions and a blood vessel;

FIG. 4 is a drawing according to the embodiment illustrating an example of a target position with respect to a maximum activity region specified by a calculating function or selected by a user;

FIG. 5 is a drawing according to the embodiment illustrating an example of setting a target position based on a center-of-gravity position of three activity regions;

FIG. 6 is a flowchart according to the embodiment illustrating an example of a procedure in an image display process;

FIG. 7 is a drawing according to the embodiment illustrating an example of displaying mutually-different hues corresponding to distances from a center position which corresponds to a position having the shortest distance from an activity region to a blood vessel;

FIG. 8 is a block diagram illustrating an exemplary configuration of an X-ray diagnosis apparatus according to a second application example of the embodiment;

FIG. 9 is a drawing according to the second application example of the embodiment illustrating an example of an image in which a placement position image is superimposed on a fluoroscopic image;

FIG. 10 is a drawing according to the second application example of the embodiment illustrating an example in which the hue of a marker corresponding to the target position has been changed; and

FIG. 11 is a flowchart according to the second application example of the embodiment illustrating an example of a procedure in a placement display process.

### DETAILED DESCRIPTION

A medical image processing apparatus according to an embodiment includes processing circuitry. The processing circuitry is configured to obtain activity position data related to positions of a plurality of activity regions that bring a site of an examined subject into an activity and a blood vessel image rendering a three-dimensional blood vessel related to the plurality of activity regions. The processing circuitry is configured to perform a registration between the activity position data and the blood vessel image. The processing circuitry is configured to calculate a positional relationship between at least one of the plurality of activity regions and the blood vessel, on the basis of a result of the registration. On the basis of the positional relationship, the processing circuitry is configured to cause a display to display information about positioning a device capable of receiving, within the blood vessel of the examined subject, electrical signals from the activity regions.

Exemplary embodiments of a medical image processing apparatus, an X-ray diagnosis apparatus, and a medical image processing method will be explained in detail below, with reference to the accompanying drawings. In the following embodiments, some of the elements referred to by using the same reference characters are assumed to perform the same operations, and duplicate explanations thereof will be omitted as appropriate.

### Embodiments

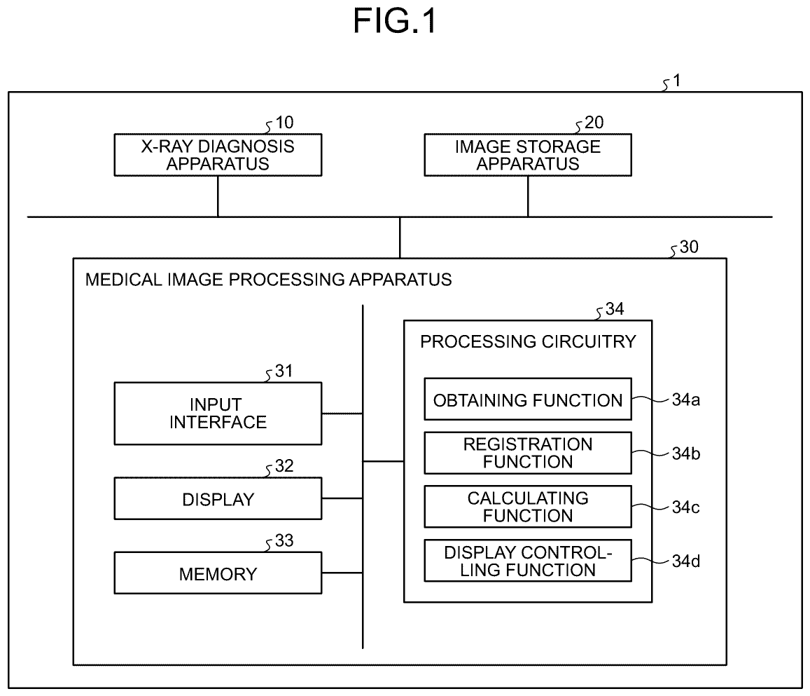

FIG. 1 is a block diagram illustrating an exemplary configuration of a medical information processing system 1 including a medical image processing apparatus 30 according to an embodiment. As illustrated in FIG. 1, the medical information processing system 1 according to the embodiment includes an X-ray diagnosis apparatus 10, an image storage apparatus 20, and a medical image processing apparatus 30. As illustrated in FIG. 1, the X-ray diagnosis apparatus 10, the image storage apparatus 20, and the medical image processing apparatus 30 are connected to each other via a network.

The X-ray diagnosis apparatus 10 is configured to acquire X-ray image data from an examined subject (hereinafter, “patient”) P. For example, the X-ray diagnosis apparatus 10 is configured to acquire a plurality of pieces of X-ray image data from the patient P and to transmit the acquired plurality of pieces of X-ray image data either to the image storage apparatus 20 or to the medical image processing apparatus 30. A configuration of the X-ray diagnosis apparatus 10 will be explained later.

The image storage apparatus 20 is configured to store therein the plurality of pieces of X-ray image data acquired by the X-ray diagnosis apparatus 10. For example, the image storage apparatus 20 is realized by using a computer machine such as a server apparatus. In the present embodiment, the image storage apparatus 20 is configured to obtain the plurality of pieces of X-ray image data from the X-ray diagnosis apparatus 10 via the network and to store the obtained plurality of pieces of X-ray image data into a memory provided either inside the apparatus or outside the apparatus.

Further, the image storage apparatus 20 is configured to store therein a functional image of the patient acquired by any of various types of diagnosis apparatuses. The functional image may be, for example, an image (hereinafter, “fMRI image”) acquired through functional Magnetic Resonance Imaging (fMRI). The fMRI image is represented by three-dimensional voxel data and corresponds, for example, to activity position data related to positions of a plurality of activity regions that bring a site of the patient into an activity. The site of the patient may be, for example, a part moved by a muscle such as a finger, any of the four limbs, a mimetic muscle, or an eyeball. Further, each of the activity regions corresponds to a region in which a hemodynamic reaction relevant to an activity of the brain or the spinal cord is visualized. When the brain of the patient is handled by the activity position data, each of the activity regions corresponds to a neural activity area of the brain. For example, the fMRI image is an image obtained by superimposing information indicating in which parts of which brain, functional activities of the brain took place, on structure information generated by performing MRI on the patient. For example, the activity position data corresponds to data expressing, by using a grayscale, the intensity of a neural activity in each of a plurality of voxels corresponding to a region of the brain. The fMRI image is generated by a magnetic resonance imaging apparatus by performing magnetic resonance imaging on the patient. Because it is possible to use a known method for generating the fMRI image, explanations thereof will be omitted.

Further, the functional image is not limited to the fMRI image described above. For example, the functional image may be a map indicating a distribution of magnitudes of myoelectric potentials acquired by an electromyograph or may be a magnetoencephalogram expressing activities of the brain of the patient in magnetic fields. Alternatively, the functional image may be a map of electrical signals acquired by an electrode sheet pasted onto the patient. In the following sections, to explain a specific example, the activity position data is assumed to be an fMRI image (voxel data). In this situation, the image storage apparatus 20 is configured to obtain the fMRI image from an MRI apparatus (not illustrated) via the network and to store the obtained fMRI image into a memory provided either inside the apparatus or outside the apparatus.

The image storage apparatus 20 is configured to store therein a blood vessel image of the patient acquired by any of various types of diagnosis apparatuses. The blood vessel image is an image rendering a three-dimensional structure of a blood vessel related to the plurality of activity regions. In other words, the blood vessel image corresponds to a 3D blood vessel model indicating the three-dimensional blood vessel structure. That is to say, the blood vessel image is volume data indicating the blood vessel structure in the vicinity of the plurality of activity regions. For example, the blood vessel image may be acquired through contrast enhanced imaging that uses an MRI apparatus, an X-ray Computed Tomography (CT) apparatus, or the X-ray diagnosis apparatus 10 configured to acquire contrast enhanced images. With the X-ray diagnosis apparatus 10 configured to acquire contrast enhanced images, the blood vessel image may be generated by implementing Digital Subtraction Angiography (DSA). Alternatively, with the X-ray diagnosis apparatus 10 configured to acquire contrast enhanced images, the blood vessel image may be generated on the basis of image processing performed on a contrast enhanced image. Because it is possible to use a known method for the blood vessel image generating processes performed by these apparatuses, explanations thereof will be omitted.

The medical image processing apparatus 30 is configured to obtain a plurality of pieces of X-ray image data in a time series from the X-ray diagnosis apparatus 10 via the network and to perform various types of processes by using the obtained plurality of pieces of X-ray image data. For example, the medical image processing apparatus 30 is realized by using a computer machine such as a workstation. In the present embodiment, the medical image processing apparatus 30 is configured to obtain the plurality of pieces of X-ray image data from either the X-ray diagnosis apparatus 10 or the image storage apparatus 20 via the network. In addition, the medical image processing apparatus 30 is configured to obtain the activity position data from the image storage apparatus 20 via the network. Further, the medical image processing apparatus 30 is configured to perform a registration between the activity position data and the blood vessel image, to calculate a positional relationship between at least one of the plurality of activity regions and the blood vessel on the basis of a result of the registration, and to cause a display 32 to display information about positioning a device capable of receiving, within the blood vessel of the patient, electrical signals from the activity regions, on the basis of the calculated positional relationship.

As illustrated in FIG. 1, the medical image processing apparatus 30 includes an input interface 31, the display 32, a memory 33, and processing circuitry 34.

The input interface 31 may be realized by using a trackball, a switch, a button, a mouse, a keyboard, a touchpad on which input operations can be performed by touching an operation surface thereof, a touch screen in which a display screen and a touchpad are integrally formed, contactless input circuitry using an optical sensor, audio input circuitry, and/or the like used for implementing various types of instructions and various types of settings. The input interface 31 is configured to convert input operations received from an operator into electrical signals and to output the electrical signals to the processing circuitry 34. Further, the input interface 31 does not necessarily need to include physical operation component parts such as the mouse, the keyboard, and/or the like. For instance, possible examples of the input interface 31 include electrical signal processing circuitry configured to receive an electrical signal corresponding to an input operation from an external input mechanism provided separately from the medical image processing apparatus 30 and to output the electrical signal to the processing circuitry 34. The input interface 31 is an example of an input unit.

The display 32 is configured to display various types of information. For example, the display 32 is configured to display a Graphical User Interface (GUI) used for receiving instructions from the operator, as well as various types of X-ray image data. For example, the display 32 may be a liquid crystal display or a Cathode Ray Tube (CRT) display. The display 32 is an example of a display unit.

For example, the memory 33 may be realized by using a semiconductor memory element such as a Random Access Memory (RAM) or a flash memory, or a hard disk, an optical disk, or the like. For example, the memory 33 is configured to store therein the plurality of pieces of X-ray image data obtained from the image storage apparatus 20. Further, for example, the memory 33 is configured to store therein programs used by pieces of circuitry included in the medical image processing apparatus 30 for realizing the functions thereof. The memory 33 is an example of a storage unit.

The processing circuitry 34 is configured to control operations of the entirety of the medical image processing apparatus 30, by executing an obtaining function 34a, a registration function 34b, a calculating function 34c, and a display controlling function 34d.

By reading and executing a program corresponding to the obtaining function 34a from the memory 33, the processing circuitry 34 is configured to obtain the blood vessel image from the image storage apparatus 20. In an example, the obtaining function 34a may be configured to obtain the blood vessel image from the X-ray diagnosis apparatus 10 implementing DAS on the patient. Further, the obtaining function 34a is configured to obtain the activity position data from the image storage apparatus 20. The obtaining function 34a is configured to store the blood vessel image and the activity position data that were obtained, into the memory 33. The processing circuitry 34 realizing the obtaining function 34a corresponds to an obtaining unit.

By reading and executing a program corresponding to the registration function 34b from the memory 33, the processing circuitry 34 is configured to perform the registration between the activity position data and the blood vessel image. For example, the registration function 34b is configured to detect a plurality of anatomical feature points (which may be referred to as anatomical landmarks) in the activity position data and the blood vessel image. To the detection of the plurality of anatomical feature points, it is possible to apply a known method such as a segmentation process or inputting data to a trained model related to image recognition. Thus, explanations thereof will be omitted. Subsequently, the registration function 34b is configured to perform the registration between the activity position data and the blood vessel image, by using the detected plurality of anatomical feature points.

For example, the registration may be a rigid body registration (e.g., an affine transformation) and/or a non-rigid body registration performed on at least one of the activity position data and the blood vessel image. As for the specific method of the registration, because it is possible to apply one of various types of known methods, explanations thereof will be omitted. By performing the registration, the registration function 34b is configured to generate an activity blood vessel superimposed image in which the activity position data is superimposed on the blood vessel image, for example, as a result of the registration performed between the activity position data and the blood vessel image. The registration function 34b is configured to store the activity position data and the blood vessel image resulting from the registration into the memory 33, as the activity blood vessel superimposed image, for example. The processing circuitry 34 realizing the registration function 34b corresponds to a registration unit.

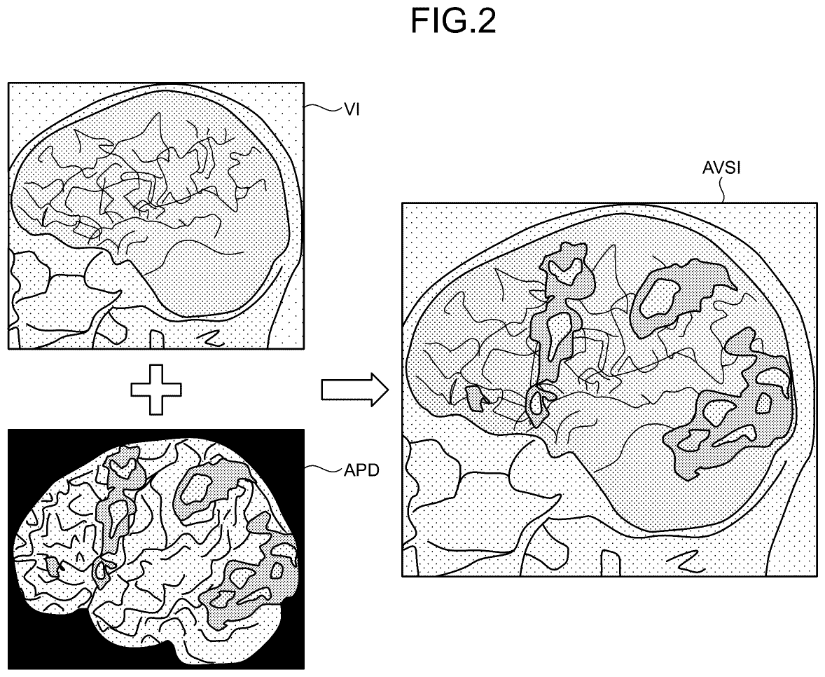

FIG. 2 is a drawing illustrating an example of an activity blood vessel superimposed image AVSI generated through a registration performed between a blood vessel image VI and activity position data APD. As illustrated in FIG. 2, the blood vessel image VI renders a three-dimensional blood vessel structure in the brain of the patient. Further, as illustrated in FIG. 2, the activity position data APD indicates activity regions corresponding to sites of the patient, by using a grayscale corresponding to magnitudes of activity amounts. The activity position data APD represents an fMRI image as illustrated in FIG. 2. Further, as illustrated in FIG. 2, in the activity blood vessel superimposed image AVSI, the activity position data APD is superimposed on the blood vessel image VI, as a result of the registration that is three-dimensional and is performed between the blood vessel image VI and the activity position data APD. The activity blood vessel superimposed image AVSI corresponds to a three-dimensional model indicating the blood vessel that is three-dimensional and the plurality of activity regions that are three-dimensional.

By reading and executing a program corresponding to the calculating function 34c from the memory 33, the processing circuitry 34 is configured to calculate a positional relationship between at least one of the plurality of activity regions and the blood vessel in the blood vessel image VI, on the basis of the result (the activity blood vessel superimposed image AVSI) of the registration performed by the registration function 34b between the activity position data and the blood vessel image VI. For example, the calculating function 34c is configured to calculate, within the three-dimensional model (the activity blood vessel superimposed image AVSI) resulting from the registration, the distance between at least one of the plurality of activity regions and the blood vessel. The processing circuitry 34 realizing the calculating function 34c corresponds to a calculating unit.

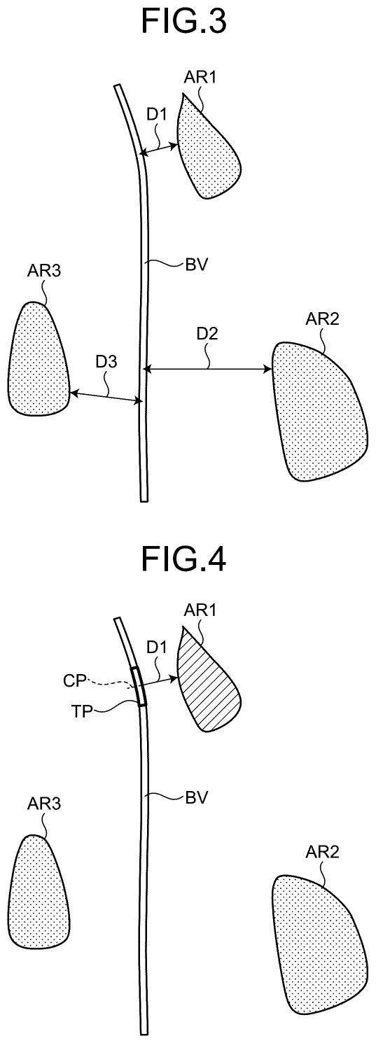

FIG. 3 is a drawing illustrating examples of distances between the plurality of activity regions and a blood vessel BV. As the plurality of activity regions, FIG. 3 illustrates three activity regions (AR1, AR2, and AR3). In the example in FIG. 3, the calculating function 34c is configured to calculate three shortest distances (D1, D2, and D3) from the blood vessel BV respectively corresponding to the three activity regions (AR1, AR2, and AR3), by using the activity blood vessel superimposed image AVSI. As illustrated in FIG. 3, among the three distances (D1, D2, and D3) related to the three activity regions (AR1, AR2, and AR3), the distance D1 related to a first activity region AR1 is shorter than the distances related to the other two activity regions (AR2 and AR3).

In the following sections, to explain a specific example, it is assumed that at least one of the plurality of activity regions is an activity region (hereinafter, “maximum activity region”) corresponding to a representative value being a maximum activity amount among the plurality of activity regions. For example, the maximum activity region corresponds to an activity region where a representative value (hereinafter, “activity representative value”) of the activity amounts is maximum, the representative value being an average value, a maximum value, or a median value of the plurality of activity amounts corresponding to a plurality of voxels in each of the plurality of activity regions. The activity representative value is calculated by the calculating function 34c by calculating the average value, the maximum value, or the median value of the plurality of activity amounts corresponding to the plurality of voxels, with respect to each of the plurality of activity regions. The maximum activity region is specified by the calculating function 34c on the basis of the activity position data. In another example, the maximum activity region may be an activity region selected by a user via the input interface 31.

More specifically, by employing the calculating function 34c, the processing circuitry 34 is configured to extract a centerline of the blood vessel BV in the activity blood vessel superimposed image AVSI. Alternatively, in place of the centerline, the calculating function 34c may extract a blood vessel wall. Because it is possible to apply a known segmentation process or the like to the extraction of the centerline and/or the blood vessel wall, explanations thereof will be omitted. Subsequently, in the activity blood vessel superimposed image AVSI, the calculating function 34c is configured to calculate the distance from the centerline or the blood vessel wall of the blood vessel BV to the maximum activity region, as the positional relationship. The distance may be the shortest distance, for example. As for activity sites of the brain in the activity position data, it is known that the site having an intense activity varies depending on a movement the patient wishes to make. For example, when it is desired to realize a plurality of goal movements by using a single stent having an electrode mounted thereon (hereinafter, “electrode-bearing stent”), it is acceptable to use a plurality of fMRI images taken at the time of making the goal movements for the calculation of the shortest distance.

By employing the calculating function 34c, the processing circuitry 34 is configured, on the basis of the positional relationship, to generate the information about the positioning of the device capable of receiving, within the blood vessel of the patient P, the electrical signals from the activity regions. The device may be, for example, a stent (an electrode-bearing stent) that can be placed in the blood vessel BV being a vein related to the activity regions and has an electrode mounted thereon for receiving the electrical signal occurring in the activity regions. In other words, the stent of the present embodiment is different from a stent used for the purpose of expanding a site (the heart, the neck, a kidney, a leg, etc.) where an artery has become narrow or a stent used when the esophagus, the trachea, or the alimentary canal is narrowed by a malignant tumor. Because it is possible to use a known stent as the electrode-bearing stent of the present embodiment, explanations thereof will be omitted.

For example, by employing the calculating function 34c, the processing circuitry 34 is configured to set, within the blood vessel BV, a position (hereinafter, “target position”) indicating a target for positioning the device in relation to receiving the electrical signal from the maximum activity region, by using the shortest distance calculated with respect to the maximum activity region. More specifically, the calculating function 34c is configured to read the length of the device from the memory 33. Subsequently, the calculating function 34c is configured to specify a position in the blood vessel BV related to the shortest distance, as the center of the device in the long-axis direction. After that, while using the specified position as the center of the device in the long-axis direction, the calculating function 34c is configured to calculate positioning (an orientation and coordinates) of the device along the blood vessel BV as the target position. For example, the target position is calculated by the calculating function 34c as three-dimensional coordinates in the activity blood vessel superimposed image AVSI. The target position, the device length, the distance between the maximum activity region and the blood vessel BV, and the like are stored in the memory 33 while being included in the information about the positioning of the device. In other words, the information about the positioning of the device includes the target position, the device length, the distance between the (maximum) activity region(s) and the blood vessel (BV), and the like.

FIG. 4 is a drawing illustrating an example of a target position TP with respect to a maximum activity region AR1 specified by the calculating function 34c or selected by the user. In FIG. 4, from among the three activity regions (AR1, AR2, and AR3), the first activity region AR1 is specified or selected as the maximum activity amount region. As illustrated in FIG. 4, in the blood vessel BV related to the maximum activity region AR1, the target position TP is set, while using the position related to a shortest distance D1 as the center (hereinafter, “center position”) CP for positioning the device.

Further, in an application example of the present embodiment, when the functional image indicates myoelectric potentials, the calculating function 34c is configured to calculate the target position in a vein related to a muscle from which the myoelectric potentials are measured. Further, although the blood vessel is in the brain in the above description, when the electrical signal passing through a nerve in the cervical vertebrae is received by the device, the blood vessel corresponds to a jugular vein, for example.

Further, when the user wishes to receive electrical signals from a plurality of activity regions, the calculating function 34c is configured to calculate a center-of-gravity position of the plurality of activity regions selected by the user via the input interface 31, for example. In this situation, the plurality of activity regions related to calculating the center-of-gravity position do not necessarily need to be selected by the user, but may be a predetermined number of higher values (e.g., n higher activity representative values as being ranked in descending order of magnitudes of the activity representative values (where n is a predetermined integer such as 2, 3, 4, or 5)) among a plurality of activity representative values corresponding to the plurality of activity regions. In another example, the plurality of activity representative values may be determined by the calculating function 34c while using a predetermined threshold value. The activity regions corresponding to activity representative values smaller than the threshold value (i.e., the activity representative values corresponding to a certain intensity or lower) shall be excluded from the calculation of the center-of-gravity position. The threshold value corresponds to the magnitudes of the activity amounts and is stored in the memory 33. Further, the user is able to adjust the threshold value as appropriate, via the input interface 31.

For example, by employing the calculating function 34c, the processing circuitry 34 is configured to calculate the center-of-gravity position of the activity amounts in the plurality of activity regions, on the basis of an activity amount of each of the plurality of voxels in each of the plurality of activity regions. More specifically, the calculating function 34c is configured to calculate the center-of-gravity position related to the plurality of activity regions, by using the activity amount of each of the plurality of voxels in each of the plurality of activity regions that are selected by the user or correspond to activity representative values exceeding the predetermined threshold value. At the time of calculating the center-of-gravity position, it is also possible to calculate the center-of-gravity position by using the magnitude of the activity amount of each of the plurality of voxels as a weight.

When the center-of-gravity position has been calculated, the processing circuitry 34 is configured, by employing the calculating function 34c, to calculate the shortest distance between the center-of-gravity position and the blood vessel BV as a positional relationship, on the basis of the activity blood vessel superimposed image AVSI which is a result of the registration. In other words, the calculating function 34c is configured to calculate the position (coordinates in the activity blood vessel superimposed image AVSI) of the blood vessel BV closest from the center-of-gravity position. Because the process of generating the information about the positioning of the device on the basis of the positional relationship was explained above, explanations will be omitted.

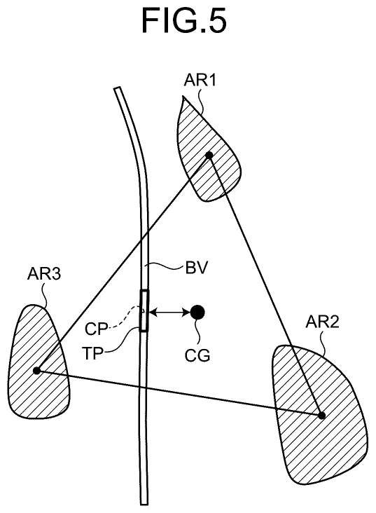

FIG. 5 is a drawing illustrating an example of setting the target position TP based on a center-of-gravity position CG of the three activity regions (AR1, AR2, and AR3). In FIG. 5, the three activity regions (AR1, AR2, and AR3) are either selected by the user or selected as regions corresponding to the activity representative values exceeding the predetermined threshold value. As illustrated in FIG. 5, in the blood vessel BV related to the three activity regions (AR1, AR2, and AR3), the center position CP related to the positioning of the device is set, while using the position related to the shortest distance from the center-of-gravity position CG of the three activity regions (AR1, AR2, and AR3) as the target position TP. In other words, the target position TP is set so that the target position TP overlaps with the center position CP.

By reading and executing a program corresponding to the display controlling function 34d from the memory 33, the processing circuitry 34 is configured to cause the display 32 to display the information about the positioning of the device (the electrode-bearing device) capable of receiving, within the blood vessel of the patient, the electrical signals from the activity regions, on the basis of the positional relationship calculated by the calculating function 34c. For example, the electrode-bearing device corresponds to an electrode-bearing stent. Accordingly, the display 32 is configured to display the information about the positioning of the electrode-bearing device. For example, the display controlling function 34d is configured to cause the display 32 to display the activity position data and the information about the positioning of the electrode-bearing device so as to be superimposed on the blood vessel image VI. At that time, the display 32 is configured to display the activity position data and the information about the positioning of the electrode-bearing device so as to be superimposed on the blood vessel image VI. The processing circuitry 34 realizing the display controlling function 34d corresponds to a display controlling unit.

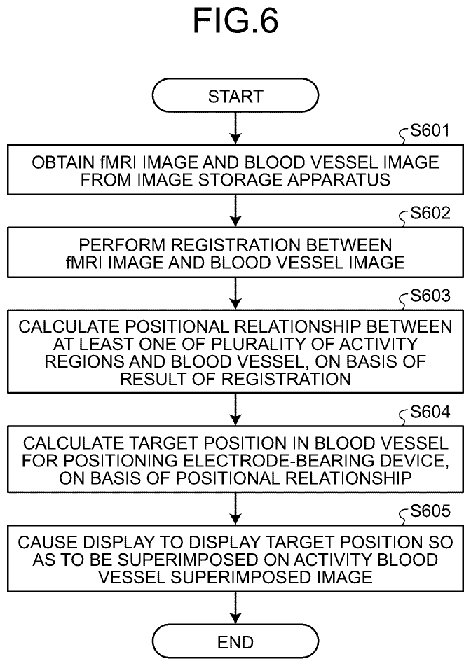

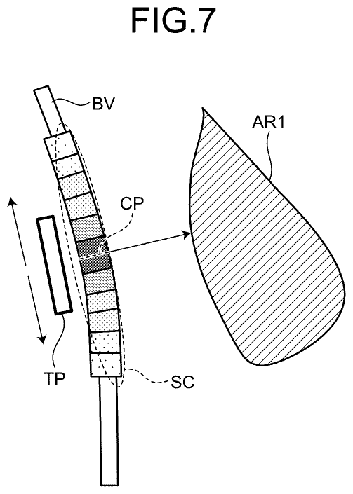

An overall configuration of the medical image processing apparatus 30 according to the embodiment has thus been explained. Next, a process (hereinafter, “image display process”) of displaying the activity position data and the information about the positioning of the electrode-bearing device so as to be superimposed on the blood vessel image VI will be explained, with reference to FIG. 6.

FIG. 6 is a flowchart illustrating an example of a procedure in the image display process. In the following sections, to explain a specific example, it is assumed that the activity position data is a three-dimensional fMRI image APD. Further, it is assumed that the blood vessel image VI is blood vessel volume data in the brain of the patient generated by the X-ray diagnosis apparatus 10 implementing a DSA method.

### The Image Display Process

### Step S601:

By employing the obtaining function 34a, the processing circuitry 34 obtains the fMRI image APD and the blood vessel image VI from the image storage apparatus 20. The fMRI image APD and the blood vessel image VI are images related to mutually the same patient. The obtaining function 34a stores the fMRI image APD and the blood vessel image VI into the memory 33.

### Step S602:

By employing the registration function 34b, the processing circuitry 34 reads the fMRI image APD and the blood vessel image VI from the memory 33. The registration 34b performs the registration between the fMRI image APD and the blood vessel image VI. The registration is a three-dimensional registration. As a result, the registration function 34b generates the activity blood vessel superimposed image AVSI. The registration function 34b stores the generated activity blood vessel superimposed image AVSI into the memory 33.

### Step S603:

By employing the calculating function 34c, the processing circuitry 34 calculates a positional relationship between at least one of the plurality of activity regions and the blood vessel BV, on the basis of the result of the registration. When one of the activity regions is selected by the user or the like, for example, when the first activity region AR1 is selected by the user as illustrated in FIG. 4, the calculating function 34c calculates, as the positional relationship, the shortest distance D1 from the blood vessel BV to the first activity region AR1, as illustrated in FIG. 4.

In another example, when a plurality of the activity regions are selected by the user or the like, for instance, when the first to the third activity regions (AR1, AR2, and AR3) are selected by the user as illustrated in FIG. 5, the calculating function 34c calculates the center-of-gravity position CG, as illustrated in FIG. 4. After that, the calculating function 34c calculates the shortest distance from the center-of-gravity position CG to the blood vessel BV.

### Step S604:

By employing the calculating function 34c, the processing circuitry 34 calculates the target position TP in the blood vessel BV for positioning the electrode-bearing device, on the basis of the calculated positional relationship. The calculating function 34c stores information (the coordinates, an orientation vector, and/or the like) related to the calculated target position TP into the memory 33.

### Step S605:

By employing the display controlling function 34d, the processing circuitry 34 causes the display 32 to display the target position TP so as to be superimposed on the activity blood vessel superimposed image AVSI. In this situation, the target position TP may be adjusted as appropriate according to a user instruction received via the input interface 31. Further, while the target position is superimposed on the activity blood vessel superimposed image AVSI, it is possible to change the selection of the activity regions as appropriate. In that situation, the processes at step S603 and thereafter are repeatedly performed. In another example, the display controlling function 34d may cause the display 32 to display the target position TP so as to be superimposed on either an fMRI image or the blood vessel image VI. When an instruction to confirm the target position TP is input as a user instruction received via the input interface 31, the display controlling function 34d stores an image (hereinafter, “target position presenting image”) obtained by superimposing the target position TP on the activity blood vessel superimposed image AVSI, into the memory 33.

The medical image processing apparatus 30 according to the embodiment described above is configured: to obtain the activity position data related to the positions of the plurality of activity regions that bring a site of the patient into an activity and the blood vessel image VI rendering the three-dimensional blood vessel BV related to the plurality of activity regions; to perform the registration between the activity position data and the blood vessel image VI that were obtained; to calculate the positional relationship between at least one of the plurality of activity regions and the blood vessel BV on the basis of the result of the registration; and to display the information about the positioning of the device capable of receiving, within the blood vessel of the patient, the electrical signals from the activity regions, on the basis of the calculated positional relationship. In this situation, in relation to the medical image processing apparatus 30 according to the present embodiment, for example, the device may be a stent having an electrode configured to receive the electrical signals occurring in the activity regions. Further, the medical image processing apparatus 30 according to the present embodiment is configured to display the activity position data and the information about the positioning of the device so as to be superimposed on the blood vessel image VI.

For example, on the basis of the result of the registration, the medical image processing apparatus 30 according to the embodiment is configured to calculate, as the positional relationship, the shortest distance D1 between one of the plurality of activity regions and the blood vessel BV. As another example, the medical image processing apparatus 30 according to the embodiment is configured to calculate the center-of-gravity position of the activity amounts in the plurality of activity regions on the basis of the activity amount of each of the plurality of voxels in each of the plurality of activity regions and to further calculate, as the positional relationship, the shortest distance D1 between the blood vessel BV and the calculated center-of-gravity position, on the basis of the result of the registration.

With these configurations, by using the medical image processing apparatus 30 according to the embodiment, on the basis of the activity position data such as the fMRI image and the blood vessel image VI, it is possible to present the user with the target position TP for placing the device, together with the activity position data and the blood vessel image VI. Consequently, by using the medical image processing apparatus 30 according to the embodiment, it is possible, in relation to the setting of the target position TP for placing the device, to present the user with an optimal device placement position while reducing user operations. As a result, by using the medical image processing apparatus 30 according to the embodiment, it is possible to enhance the efficiency (a throughput) of diagnosing processes related to placing the device.

### First Application Example

In the present application example, a change to the target position is presented when the target position TP calculated in the embodiment is not suitable for the placement of the device. For example, by employing the calculating function 34c, the processing circuitry 34 is configured to calculate a dangerous position having a danger in relation to the positioning of the electrode-bearing device, on the basis of structure information of the blood vessel BV in the blood vessel image VI. For example, the structure information of the blood vessel BV corresponds to curvature of the blood vessel BV overlapping with the target position TP and whether or not the blood vessel BV overlapping with the target position TP has a branch. For example, the calculating function 34c is configured to calculate the curvature of the blood vessel BV overlapping with the target position TP. Alternatively, in place of the curvature, the calculating function 34c may be configured to calculate a curvature radius of the blood vessel BV overlapping with the target position TP. Further, the calculating function 34c is configured to specify whether or not the blood vessel BV overlapping with the target position TP has a branch. The image display process related to the present application example is performed at step S605 and thereafter, for example.

By employing the calculating function 34c, the processing circuitry 34 is configured to calculate the dangerous position (e.g., coordinates in the blood vessel image VI) having a danger in relation to the positioning of the electrode-bearing device, on the basis of the length of the electrode-bearing device, elasticity of the electrode-bearing device, the calculated curvature (or the calculated curvature radius), and whether or not there is a branch. For example, the calculating function 34c may be configured to determine the target position TP as a dangerous position, by calculating the length of an overlapping part between the length of the electrode-bearing device and the length of the branch. The dangerous position corresponds to the situation where the blood vessel BV overlapping with the target position TP has a branch, i.e., the situation where a part of the electrode-bearing device overlaps with the branch, when the electrode-bearing device is positioned at the target position TP.

When the type of the electrode-bearing device is selected via the input interface 31, the length and the elasticity of the electrode-bearing device are determined by the calculating function 34c, for example, by comparing the selected type with a correspondence table keeping different types in correspondence with lengths and elasticity values of electrode-bearing devices. Further, the processing circuitry 34, the calculating function 34c is configured to calculate the dangerous position for the target position TP, on the basis of the curvature (or the curvature radius) of the blood vessel BV, the length of the electrode-bearing device, and the elasticity of the electrode-bearing device. The calculating function 34c is configured to store the calculated dangerous position (coordinates) into the memory 33.

By employing the display controlling function 34d, the processing circuitry 34 is configured to cause the display 32 to display, over the blood vessel rendered in the blood vessel image VI, the positioning of the device among the information about the positioning of the device, by using mutually-different hues in accordance with distances from the position (the abovementioned center position CP) related to the shortest distance. In this situation, the display 32 is configured to display, over the blood vessel rendered in the blood vessel image VI, the positioning of the electrode-bearing device by using the mutually-different hues in accordance with the distances from the center position CP.

FIG. 7 is a drawing illustrating an example of displaying mutually-different hues SC corresponding to the distances from the center position CP which corresponds to the position having the shortest distance from the activity region AR1 to the blood vessel BV. In FIG. 7, the mutually-different hues SC are expressed with different levels of darkness of the hatching. For example, as illustrated in FIG. 7, the display 32 is configured to display the blood vessel BV by using the mutually-different hues SC that gradually change every millimeter away from the center position SC. As illustrated in FIG. 7, when the target position TP is moved/adjusted along the blood vessel BV according to a user operation received via the input interface 31, the mutually-different hues SC corresponding to the distances from the center position CP correspond to a scale.

Further, by employing the display controlling function 34d, the processing circuitry 34 is configured to cause the display 32 to display the calculated dangerous position over the blood vessel BV by using a hue (hereinafter, “danger hue”) different from the abovementioned hues corresponding to the scale. The danger hue is the hue different from the hues illustrated in FIG. 7 and may be an alert color such as red or orange, for example. In this situation, in accordance with the location of the target position TP in the blood vessel BV, the display controlling function 34d may be configured to cause the display 32 to display curvature or a curvature radius in the location. As a result, the user is able to understand the curvature or the curvature radius at the target position TP.

The medical image processing apparatus 30 according to the first application example of the embodiment described above is configured to display, over the blood vessel BV rendered in the blood vessel image VI, the positioning of the device among the information about the positioning of the device, by using the mutually-different hues CS in accordance with the distances from the position (the center position CP) related to the shortest distance. With this configuration, by using the medical image processing apparatus 30 according to the first application example of the embodiment, the user is able to conveniently move/adjust the target position TP via the input interface 31.

Further, the medical image processing apparatus 30 according to the first application example of the embodiment is configured to calculate, on the basis of the structure information of the blood vessel BV, the dangerous position having a danger in relation to the positioning of the device and to further display, over the blood vessel BV, the dangerous position by using the hue (the danger hue) different from the abovementioned hues. With this configuration, when the curvature of the blood vessel BV at the calculated target position TP is large or when the target position TP is not suitable for the placement of the electrode-bearing device due to being a site (e.g., a branch) considered to have another type of danger, the medical image processing apparatus 30 according to the first application example of the embodiment is able to display the danger hue over the blood vessel BV. As a result, with a single motion via the input interface 31, the user is able to change the target position TP to a position where the placement of the electrode-bearing device is safe. Because the other advantageous effects are the same as those of the embodiment, explanations thereof will be omitted.

### Second Application Example

In the present application example, the target position TP determined through the input interface 31 is displayed on the display together with a fluoroscopic image obtained from the X-ray diagnosis apparatus 10. In the following sections, to explain a specific example, it is assumed in the present application example that the various types of functions of the processing circuitry 34 in the medical image processing apparatus 30 are provided in the X-ray diagnosis apparatus 10. Alternatively, the technical features realized in the present application example may be realized by the medical image processing apparatus 30 alone, by obtaining the fluoroscopic image from the X-ray diagnosis apparatus 10.

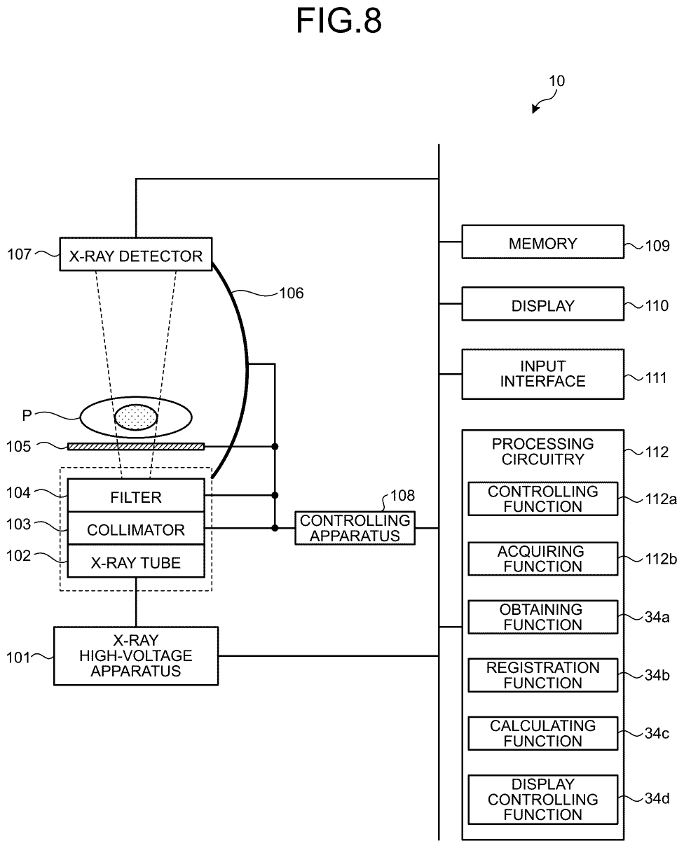

FIG. 8 is a block diagram illustrating an exemplary configuration of the X-ray diagnosis apparatus 10 according to a second application example of the embodiment. As illustrated in FIG. 8, the X-ray diagnosis apparatus 10 includes an X-ray high-voltage apparatus 101, an X-ray tube 102, a collimator 103, a filter 104, a tabletop 105, a C-arm 106, an X-ray detector 107, a controlling apparatus 108, a memory 109, a display 110, an input interface 111, and processing circuitry 112.

Under control of the processing circuitry 112, the X-ray high-voltage apparatus 101 is configured to supply high voltage to the X-ray tube 102. For example, the X-ray high-voltage apparatus 101 includes: a high-voltage generating apparatus having electric circuitry such as a transformer and a rectifier and being configured to generate the high voltage to be applied to the X-ray tube 102; and an X-ray controlling apparatus configured to control output voltage corresponding to X-rays to be emitted by the X-ray tube 102. In this situation, the high-voltage generating apparatus may be of a transformer type or an inverter type.

The X-ray tube 102 is a vacuum tube having a negative pole (a filament) configured to generate thermo electrons and a positive pole (a target) configured to generate X-rays in response to collision of the thermo electrons thereon. The X-ray tube 102 is configured to generate the X-rays, by causing the thermo electrons to be emitted from the negative pole toward the positive pole, with application of the high voltage supplied from the X-ray high-voltage apparatus 101. In this manner the X-ray tube 102 is configured to emit the X-rays onto the patient P.

The collimator (which may be referred to as an X-ray limiting apparatus) 103 includes, for example, four slidable limiting blades. By sliding the limiting blades, the collimator 103 is configured to narrow down the X-rays generated by the X-ray tube 102 so as to be emitted onto the patient P. In this situation, the limiting blades are plate-like members structured by using lead or the like and are provided in the vicinity of an X-ray emission opening of the X-ray tube 102 so as to adjust an emission range of the X-rays.

For the purpose of reducing radiation exposure of the patient P and enhancing image quality of X-ray image data, the filter 104 is configured to change radiation quality of passing X-rays by using the materials and/or the thickness thereof, so as to reduce soft-ray components that can easily be absorbed by the patient P and to reduce high-energy components that may lead to degradation of the contrast of the X-ray image data. Further, the filter 104 is configured to change radiation amounts and the emission range of the X-rays by using the materials, the thickness, and/or the position thereof, to attenuate the X-rays so that the X-rays emitted from the X-ray tube 102 onto the patient P have a predetermined distribution.

The tabletop 105 is a bed on which the patient P is placed and is disposed over a table (not illustrated). It should be noted that the patient P is not included in the X-ray diagnosis apparatus 10.

The C-arm 106 is configured to hold the X-ray tube 102, the collimator 103, the filter 104, and the X-ray detector 107, so that the first three elements oppose the X-ray detector 107 while the patient P is interposed therebetween. Although FIG. 8 illustrates an example in which the X-ray diagnosis apparatus 10 is of a single-plane type, possible embodiments are not limited to this example, and the X-ray diagnosis apparatus 10 may be of a biplane type. Further, to a driving mechanism related to the C-arm 106, it is possible to apply a known driving mechanism such as a mechanism related to angiography apparatus for circulatory organs, for example.

For example, the X-ray detector 107 is an X-ray Flat Panel Detector (FPD) including detecting elements arranged in a matrix formation. The X-ray detector 107 is configured to detect X-rays that were emitted from the X-ray tube 102 and have passed through the patient P and to output a detection signal corresponding to a detected X-ray amount to the processing circuitry 112. In this situation, the X-ray detector 107 may be an indirect-conversion type detector including a grid, a scintillator array, and an optical sensor array or may be a direct-conversion type detector including a semiconductor element configured to convert incident X-rays into an electrical signal.

The controlling apparatus 108 includes a driving mechanism such as a motor and an actuator or the like and circuitry configured to control the mechanism. Under control of the processing circuitry 112, the controlling apparatus 108 is configured to control operations of the collimator 103, the filter 104, the tabletop 105, the C-arm 106, and the like. For example, the controlling apparatus 108 is configured to control the emission range of the X-rays emitted onto the patient P, by adjusting an opening degree of the limiting blades in the collimator 103. Further, the controlling apparatus 108 is configured to control a distribution of radiation amounts of the X-rays emitted onto the patient P, by adjusting the position of the filter 104. Further, for example, the controlling apparatus 108 is configured to rotate/move the C-arm 106 and to move the tabletop 105.

For example, the memory 109 may be realized by using a semiconductor memory element such as a RAM or a flash memory, or a hard disk, an optical disk, or the like. For example, the memory 109 is configured to receive and store therein the X-ray image data acquired by the processing circuitry 112. Further, the memory 109 is configured to store therein programs that correspond to various types of functions and are read and executed by the processing circuitry 112. In this situation, the memory 109 has stored therein the same storage contents as what is stored in the memory 33 of the medical image processing apparatus 30 illustrated in FIG. 1.

The display 110 is configured to display various types of information. For example, the display 110 is configured to display a GUI used for receiving instructions from an operator and various types of X-ray image data. For example, the display 110 may be a liquid crystal display or a CRT display. In this situation, the display 110 is configured to display the same display contents as what is displayed by the display 32 of the medical image processing apparatus 30 illustrated in FIG. 1.

The input interface 111 may be realized by using a trackball, a switch, a button, a mouse, a keyboard, a touchpad on which input operations can be performed by touching an operation surface thereof, a touch screen in which a display screen and a touchpad are integrally formed, contactless input circuitry using an optical sensor, audio input circuitry, and/or the like used for implementing various types of instructions and various types of settings. The input interface 111 is configured to convert input operations received from the operator into electrical signals and to output the electrical signals to the processing circuitry 112. Further, the input interface 111 does not necessarily need to include physical operation component parts such as the mouse, the keyboard, and/or the like. For instance, possible examples of the input interface 111 include electrical signal processing circuitry configured to receive an electrical signal corresponding to an input operation from an external input mechanism provided separately from the X-ray diagnosis apparatus 10 and to output the electrical signal to the processing circuitry 112. In this situation, the input interface 111 is capable of receiving inputs of the same operations as those received by the input interface 31 of the medical image processing apparatus 30 illustrated in FIG. 1.

The processing circuitry 112 is configured to control operations of the entirety of the X-ray diagnosis apparatus 10, by executing a controlling function 112a, an acquiring function 112b, the obtaining function 34a, the registration function 34b, the calculating function 34c, and the display controlling function 34d. Among the processes related to the obtaining function 34a, the registration function 34b, the calculating function 34c, and the display controlling function 34d, explanations of some of the processes that are the same as those in the embodiment will be omitted.

By reading and executing a program corresponding to the controlling function 112a from the memory 109, the processing circuitry 112 is configured to control the various types of functions of the processing circuitry 112, on the basis of input operations received from the operator via the input interface 111. The processing circuitry 112 realizing the controlling function 112a corresponds to a controlling unit.

By reading and executing a program corresponding to the acquiring function 112b from the memory 109, the processing circuitry 112 is configured to acquire the X-ray image data. For example, by controlling the X-ray high-voltage apparatus 101 so as to adjust the voltage supplied to the X-ray tube 102, the acquiring function 112b is configured to control the amount and the on/off of the X-rays to be emitted onto the patient P. Further, by controlling the controlling apparatus 108 so as to adjust the opening degree of the limiting blades included in the collimator 103, the acquiring function 112b is configured to control the emission range of the X-rays to be emitted onto the patient P. Furthermore, the acquiring function 112b is configured to control the controlling apparatus 108 so as to control the distribution of the radiation amounts of the X-rays by adjusting the position of the filter 104.

Further, by employing the acquiring function 112b, the processing circuitry 112 is configured to control the controlling apparatus 108 so as to control the rotation and the moving of the C-arm 106, the moving of the tabletop 105, and the like. Further, the acquiring function 112b is configured to generate the X-ray image data on the basis of the detection signal received from the X-ray detector 107 and to store the generated X-ray image data into the memory 109. In this situation, the acquiring function 112b may be configured to perform various types of image processing processes on the X-ray image data stored in the memory 109. For example, the acquiring function 112b may be configured to perform a noise reduction process and/or a scattered ray correction using image processing filters on the X-ray image data. The processing circuitry 112 realizing the acquiring function 112b corresponds to an acquiring unit. With these configurations, the acquiring function 112b is configured to generate the blood vessel image VI rendering the three-dimensional blood vessel BV, on the basis of the emission of the X-rays onto the patient P. In this situation, the processing circuitry 112 realizing the acquiring function 112b corresponds to an image generating unit.

By employing the obtaining function 34a, the processing circuitry 112 is configured to obtain the fluoroscopic image related to the patient P, from the acquiring function 112b. Further, the obtaining function 34a is configured to obtain, from the controlling apparatus 108, geometric information of an imaging system relevant to the obtained fluoroscopic image and related to imaging the patient P. For example, the geometric information may be information related to geometry such as the position of the C-arm 106, an angle of the C-arm 106, the position (coordinates) of the tabletop 105, and/or the like. As another example, the geometric information may information about geometry related to the X-ray tube 102 and the X-ray detector 107 used in the X-ray imaging performed on the patient P. The obtaining function 34a may be configured to store the fluoroscopic image and the geometric information into the memory 109 so as to be kept in association with each other. Further, the obtaining function 34a is configured to obtain, from the memory 109, the blood vessel image VI (hereinafter, “placement position setting image”) set with the target position TP.

By employing the registration function 34b, the processing circuitry 112 is configured to perform a registration between the placement position setting image and the fluoroscopic image. On the basis of a result of the registration, the registration function 34b is configured to bring the placement position setting image into association with the geometric information related to the fluoroscopic image. The registration function 34b is configured to store, into the memory 109, information (hereinafter, “association information”) about the association between the placement position setting image and the geometric information. The association information may be, for example, a matrix used for displaying, on the basis of the geometric information, the placement position setting image with the same viewpoint and from the same line-of-sight direction as those of the fluoroscopic image.

By employing the calculating function 34c, the processing circuitry 112 is configured, on the basis of the geometric information related to the obtained fluoroscopic image, to calculate a target position TP indicating the target for positioning the device among the information about the positioning of the device, pursuant to changes in the geometric information of the imaging system. More specifically, by using the geometric information related to the obtained fluoroscopic image and the association information, the calculating function 34c is configured to transform the placement position setting image resulting from the registration into an image corresponding to the viewpoint and the line-of-sight direction that are the same as those of the fluoroscopic image. With these configurations, the calculating function 34c is configured to calculate a display-purpose image (hereinafter, “placement position image”) of the placement position setting image corresponding to the fluoroscopic image, as a result of the transformation of the placement position setting image realized pursuant to the changes in the geometric information of the imaging system.

By reading and executing a program corresponding to the display controlling function 34d from the memory 109, the processing circuitry 112 is configured to cause the display 110 to display the X-ray image data acquired by the acquiring function 112b. Further, the display controlling function 34d is configured to cause the display 110 to display a GUI used for receiving instructions from the operator.

Further, by employing the display controlling function 34d, the processing circuitry 112 is configured to cause the display 110 to display the target position TP so as to be superimposed on the fluoroscopic image. In other words, the display controlling function 34d is configured to cause the display 110 to display the placement position image so as to be superimposed on the fluoroscopic image. In this situation, the display controlling function 34d may be configured to cause the display 110 to display the target position TP in the placement position image so as to be superimposed on the fluoroscopic image. With these configurations, the display 110 is configured to display the target position TP so as to be superimposed on the fluoroscopic image. As another example, the display controlling function 34d may be configured to cause a part of the display 110 displaying the fluoroscopic image or a monitor (e.g., a reference monitor not illustrated in FIG. 8) different from the display 110 displaying the fluoroscopic image to display a display image corresponding to at least one of FIGS. 3 to 5 and FIG. 7.

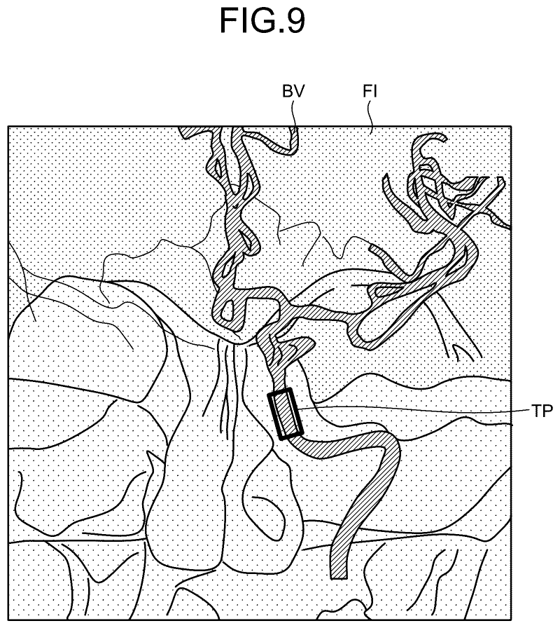

FIG. 9 is a drawing illustrating an example of an image in which the placement position image is superimposed on a fluoroscopic image FI. As illustrated in FIG. 9, in the fluoroscopic image FI of the patient P, the target position TP is displayed over the blood vessel BV. In this situation, the display 110 displays the blood vessel BV and the target position TP as being updated in accordance with updates of the fluoroscopic image.

When the electrode-bearing device has moved up to the target position TP, the processing circuitry 112 is configured, by employing the display controlling function 34d, to cause the display 110 to display the target position TP by changing the hue of the marker thereof. For example, the calculating function 34c is configured to calculate the position of the electrode-bearing device in the fluoroscopic image FI. More specifically, by using a known image recognition technique, the calculating function 34c is configured to specify the position of the electrode-bearing device in the fluoroscopic image FI. Subsequently, the calculating function 34c is configured to calculate the difference between the specified position of the electrode-bearing device and the target position TP. The difference may be, for example, the difference (the distance) between coordinates of the specified position of the electrode-bearing device and coordinates of the target position TP. By comparing the calculated difference with a value set in advance, the calculating function 34c is configured to determine that the electrode-bearing device has reached the target position TP, when the calculated difference is equal to or smaller than the value set in advance. The value set in advance is stored in the memory 109. In response to the determination that the electrode-bearing device has reached the target position TP, the display controlling function 34d is configured to cause the display 110 to display the target position TP by changing the hue of the marker thereof.

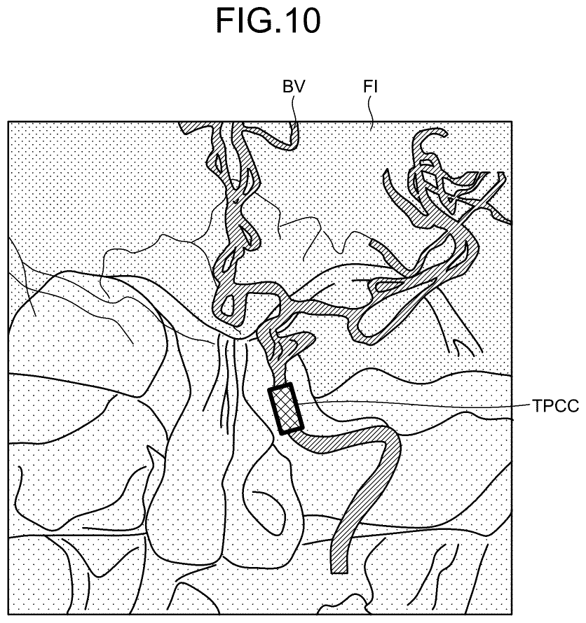

FIG. 10 is a drawing illustrating an example in which the hue of a marker TPCC corresponding to the target position TP has been changed. As illustrated in FIG. 10, when the electrode-bearing device has reached the target position TP, the display 110 displays the marker TPCC by changing the hue thereof (indicated with hatching in FIG. 10), in a manner different from FIG. 9. In other words, the display 110 is configured to notify the user that the electrode-bearing device has reached the target position TP, as being triggered by the electrode-bearing device reaching the target position TP (hereinafter, “reaching of the device”). The notification about the reaching of the device does not necessarily need to be realized with changing the hue of the marker indicating the target position TP and may be realized with audio or turning on a light, for example. When the notification about the reaching of the device is output as audio, the audio does not necessarily need to be output from the display 110 and may be output from a known speaker or the like.

In the X-ray diagnosis apparatus 10 illustrated in FIG. 8, the processing functions are stored in the memory 109 in the form of computer-executable programs. The processing circuitry 112 is a processor configured to realize the functions corresponding to the programs by reading and executing the programs from the memory 109. In other words, the processing circuitry 112 that has read the programs has the functions corresponding to the read programs. Further, although the example was explained with reference to FIG. 8 in which the single piece of processing circuitry (the processing circuitry 112) realizes the controlling function 112a, the acquiring function 112b, the obtaining function 34a, the registration function 34b, the calculating function 34c, and the display controlling function 34d, it is also acceptable to structure the processing circuitry 112 by combining together a plurality of independent processors, so that the functions are realized as a result of the processors executing the programs.

The term “processor” used in the above explanations denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)). The processors of the processing circuitry 34 and the processing circuitry 112 are configured to realize the functions by reading and executing the programs saved in the memory 33 and the memory 109, respectively. In this situation, instead of having the programs saved in the memory 33 and the memory 109, it is also acceptable to directly incorporate the programs in the circuitry of the processors. In that situation, the processors realize the functions by reading and executing the programs incorporated in the circuitry thereof. Further, the processors of the present embodiments do not each necessarily have to be structured as a single piece of circuitry. It is also acceptable to structure one processor by combining together a plurality of pieces of independent circuitry so as to realize the functions thereof.

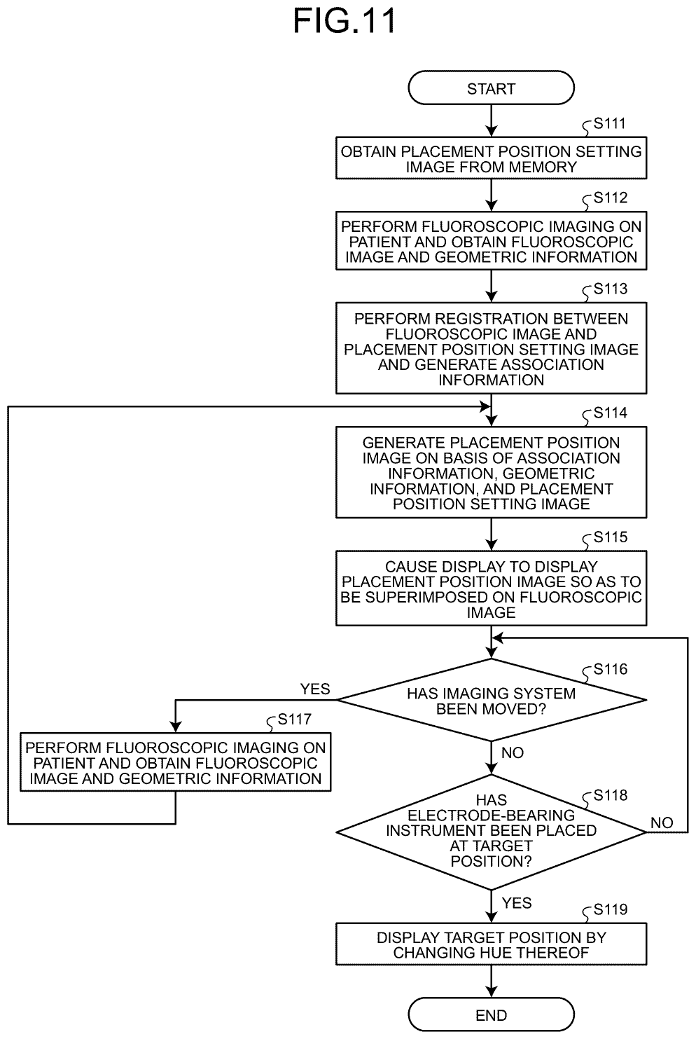

An overall configuration of the X-ray diagnosis apparatus 10 according to the second application example of the embodiment has thus been explained. Next, a display process (hereinafter, “placement display process”) performed at the time of placing the electrode-bearing device at the target position TP will be explained, with reference to FIG. 11. FIG. 11 is a flowchart illustrating an example of a procedure in the placement display process. For example, the placement display process is a process of causing the display 110 to display the blood vessel BV and the target position TP so as to be superimposed on the fluoroscopic image pursuant to the moving of the imaging system, in accordance with updates of the fluoroscopic image made along with the moving of the imaging system.

### The Placement Display Process

### Step S111:

By employing the obtaining function 34a, the processing circuitry 112 obtains the placement position setting image from the memory 109.

### Step S112:

Under the control of the controlling function 112a, the controlling apparatus 108 performs fluoroscopic imaging on the patient P. Accordingly, the processing circuitry 112 generates a fluoroscopic image by employing the acquiring function 112b. In this situation, the geometric information is kept in association with the fluoroscopic image. By employing the obtaining function 34a, the processing circuitry 112 obtains the fluoroscopic image and the geometric information.

### Step S113:

By employing the registration function 34b, the processing circuitry 112 performs a registration between the placement position setting image and the fluoroscopic image. The registration corresponds to bringing the line of sight and the line-of-sight direction of the placement position setting image into association with the geometric information of the fluoroscopic image. As a result, the registration function 34b generates the association information keeping the fluoroscopic image in association with the placement position setting image.

### Step S114:

By employing the calculating function 34c, the processing circuitry 112 generates a placement position image on the basis of the association information, the geometric information, and the placement position setting image. The placement position image is an image generated with a viewpoint and a line-of-sight direction that are the same as those of the fluoroscopic image and includes the blood vessel BV and the target position TP.

### Step S115:

By employing the display controlling function 34d, the processing circuitry 112 causes the display 110 to display the placement position image so as to be superimposed on the fluoroscopic image. As a result, the display 110 displays the target position TP over the blood vessel BV within the fluoroscopic image FI of the patient P, as illustrated in FIG. 9. In this situation, the display controlling function 34d may cause a part of the display 110 or a reference monitor or the like different from the display 110 to display a display image corresponding to at least one of FIGS. 3 to 5 and FIG. 7 and/or an fMRI image.

### Step S116:

When the imaging system (the C-arm 106, the tabletop 105, etc.) is moved during the fluoroscopic imaging performed on the patient P (step S116: Yes), the process at step S117 will be performed. On the contrary, when the imaging system is not moved during the fluoroscopic imaging performed on the patient P (step S116: No), the process at step S118 will be performed.

### Step S117:

Under the control of the controlling function 112a, the controlling apparatus 108 performs the fluoroscopic imaging on the patient P. As a result, by employing the acquiring function 112b, the processing circuitry 112 generates a fluoroscopic image. By employing the obtaining function 34a, the processing circuitry 112 obtains the fluoroscopic image and the geometric information. After the present step, the processes at step S114 and thereafter will be repeatedly performed.

### Step S118:

By employing the calculating function 34c, the processing circuitry 112 judges whether or not the electrode-bearing device has reached the target position TP, on the basis of the difference between the position of the electrode-bearing device in the fluoroscopic image FI and the target position Tp. When the electrode-bearing device has been placed at the target position TP (step S118: Yes), the process at step S119 will be performed. On the contrary, when the electrode-bearing device has not been placed at the target position TP (step S118: No), the processes at step S116 and thereafter will be performed.

### Step S119:

By employing the display controlling function 34d, the processing circuitry 112 causes the display 110 to display the target position TP by changing the hue of the marker thereof. In this situation, as illustrated in FIG. 10, the display 110 displays the marker TPCC by changing the hue thereof (the hatching in FIG. 10) from the hue used before the electrode-bearing device reached the target position TP. The reaction at the time of the electrode-bearing device reaching the target position TP does not necessarily need to be the changing of the hue of the marker TPCC and may be an output of audio, for example. The placement display process ends with the present step.

As a modification example of the second application example described above, an image corresponding to at least one of FIGS. 3 to 5 and FIG. 7 related to the activity blood vessel superimposed image may be used in the placement display process, similarly to the placement position setting image in the placement display process. In that situation, as the image corresponding to at least one of FIGS. 3 to 5 and FIG. 7 related to the activity blood vessel superimposed image, the calculating function 34c is configured to generate an image corresponding to the same viewpoint and the same line-of-sight direction as those of the fluoroscopic image pursuant to the moving of the imaging system, similarly to the manner described in the placement display process. Subsequently, the display controlling function 34d is configured to cause a part of the display 110, a reference monitor, or the like to display the image generated pursuant to the moving of the imaging system. With these configurations, the part of the display 110, the reference monitor, or the like is configured to display the image that corresponds to at least one of FIGS. 3 to 5 and FIG. 7 and corresponds to the fluoroscopic image, pursuant to the moving of the imaging system, as a reference image for the manipulation.

Further, the placement display process is not limited to using the geometric information. Alternatively, it is also acceptable to generate a placement position image or the like, by performing, in accordance with the moving of the imaging system, a registration (an image processing process) between the fluoroscopic image and the placement position setting image, pursuant to the moving of the imaging system, for example. In that situation, the geometric information obtaining process is unnecessary.

The X-ray diagnosis apparatus 10 according to the second application example of the embodiment described above is configured: to emit the X-rays onto the patient P; to generate the blood vessel image VI rendering the three-dimensional blood vessel BV on the basis of the emission of the X-rays onto the patient P; to obtain the activity position data related to the positions of the plurality of activity regions that bring a site of the patient P into an activity and the blood vessel image VI rendering the three-dimensional blood vessel BV related to the plurality of activity regions; to perform the registration between the activity position data and the blood vessel image VI that were obtained; to calculate the positional relationship between at least one of the plurality of activity regions and the blood vessel BV on the basis of a result of the registration; and to display the information about the positioning of the device capable of receiving, within the blood vessel of the patient P, the electrical signals from the activity regions, on the basis of the calculated positional relationship. Further, the X-ray diagnosis apparatus 10 according to the second application example of the embodiment is configured: to obtain the fluoroscopic image FI generated on the basis of the fluoroscopic imaging performed on the patient P and the geometric information of the imaging system related to imaging the patient P; to calculate the target position TP indicating the target for the positioning of the electrode-bearing device among the information about the positioning of the electrode-bearing device pursuant to the changes in the geometric information of the imaging system, on the basis of the geometric information related to the fluoroscopic image FI; and to cause the display 110 to display the target position TP so as to be superimposed on the fluoroscopic image FI.

With these configurations, by using the X-ray diagnosis apparatus 10 according to the second application example of the embodiment, because the three-dimensional blood vessel image VI is used as a base, the user is able to recognize the current position of the target position TP in relation to the depth and the up-and-down and left-and-right directions of the blood vessel BV, pursuant to the rotation and the moving of the C-arm 106 and the tabletop 105, similarly to a display of a three-dimensional roadmap image. Further, because the X-ray diagnosis apparatus 10 according to the second application example of the embodiment is able to simultaneously display the display image corresponding to at least one of FIGS. 3 to 5 and FIG. 7 on the reference monitor, for example. Accordingly, the user is able to compare and/or check these images.

Further, the X-ray diagnosis apparatus 10 according to the second application example of the embodiment is configured to notify that the electrode-bearing device has reached the target position TP, as being triggered by the electrode-bearing device reaching the target position TP. For example, when the electrode-bearing device has been moved up to the goal position TP, the X-ray diagnosis apparatus 10 according to the second application example of the embodiment is able to change the color of the marking of the placement position for the electrode-bearing device, in the situation where the electrode-bearing device has accurately reached the target position TP. With this configuration, the X-ray diagnosis apparatus 10 according to the second application example of the embodiment is able to give the user feedback indicating (to notify the user) that the electrode-bearing device has reached the goal position TP. Because the other advantageous effects are the same as those of the embodiment, explanations thereof will be omitted.

When the technical concept of the present embodiment is realized as a medical image processing method, the medical image processing method includes: obtaining activity position data related to positions of a plurality of activity regions that bring a site of a patient P into an activity and a blood vessel image VI rendering a three-dimensional blood vessel BV related to the plurality of activity regions; performing a registration between the activity position data and the blood vessel image VI; calculating a positional relationship between at least one of the plurality of activity regions and the blood vessel BV, on the basis of a result of the registration; and displaying, on the basis of the positional relationship, information about positioning an device capable of receiving, within the blood vessel of the patient P, electrical signals from the activity regions. Further, the medical image processing method may further include: obtaining a fluoroscopic image FI generated on the basis of fluoroscopic imaging performed on the patient P and geometric information of an imaging system related to imaging the patient P; calculating, on the basis of the geometric information related to the fluoroscopic image FI, a target position TP indicating a target for positioning the device among the information about the positioning of the device, pursuant to a change in the geometric information of the imaging system; and displaying the target position TP so as to be superimposed on the fluoroscopic image FI. The processing procedures in the medical image processing method are compliant with the image display process and the placement display process. Further, advantageous effects of the medical image processing method are the same as those of the embodiment, the first application example, and the second application example. Thus, explanations of the processing procedures and the advantageous effects of the medical image processing method will be omitted.

When the technical concept of the embodiment is realized as a medical image processing program, the medical image processing program causes a computer to realize: obtaining activity position data related to positions of a plurality of activity regions that bring a site of a patient P into an activity and a blood vessel image VI rendering a three-dimensional blood vessel BV related to the plurality of activity regions; performing a registration between the activity position data and the blood vessel image VI; calculating a positional relationship between at least one of the plurality of activity regions and the blood vessel BV, on the basis of a result of the registration; and displaying, on the basis of the positional relationship, information about positioning a device capable of receiving, within the blood vessel of the patient P, electrical signals from the activity regions. Further, the medical image processing program may cause the computer to further realize: obtaining a fluoroscopic image FI generated on the basis of fluoroscopic imaging performed on the patient P and geometric information of an imaging system related to imaging the patient P; calculating, on the basis of the geometric information related to the fluoroscopic image FI, a target position TP indicating a target for positioning the device among the information about the positioning of the device, pursuant to a change in the geometric information of the imaging system; and displaying the target position TP so as to be superimposed on the fluoroscopic image FI.

For example, it is also possible to realize the image display process and the placement display process by installing an image evaluation program in a computer of the medical image processing apparatus 30 or the X-ray diagnosis apparatus 10 illustrated in FIG. 1 and loading the program into a memory. In that situation, it is also possible to store and distribute the program capable of causing the computer to execute the processes in a storage medium such as a magnetic disk (e.g., a hard disk), an optical disk (e.g., a Compact Disk Read-Only Memory (CD-ROM), a Digital Versatile Disk (DVD)), or a semiconductor memory. Further, the medical image processing program does not necessarily need to be distributed via the abovementioned media and may be distributed by using an electric communication function, such as being downloaded via the Internet, for example. The processing procedures of the medical image processing program are compliant with the image display process and the placement display process. Further, advantageous effects of the medical image processing program are the same as those of the embodiment, the first application example, and the second application example. Thus, explanations of the processing procedures and the advantageous effects of the medical image processing program will be omitted.

According to at least one aspect of the embodiments, the first application example, the second application example, and the like described above, it is possible to provide the user with the information about the positioning of the device capable of receiving the electrical signals from the activity regions that bring a site of the patient P into an activity. Consequently, according to at least one aspect of the embodiments, the first application example, the second application example, and the like, it is possible to accurately and safely place the device by calculating, on the apparatus side, an optimal device placement position on the basis of the positional relationship between a neural activity area of the brain and a blood vessel, for example.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising:
processing circuitry configured
to obtain activity position data related to positions of a plurality of activity regions that bring a site of an examined subject into an activity and a blood vessel image rendering a three-dimensional blood vessel related to the plurality of activity regions;
to perform a registration between the activity position data and the blood vessel image; and
to calculate a positional relationship between at least one of the plurality of activity regions and the blood vessel, on a basis of a result of the registration, and
to cause a display to display, on a basis of the positional relationship, information about positioning a device capable of receiving, within the blood vessel of the examined subject, electrical signals from the activity regions.

2. The medical image processing apparatus according to claim 1, wherein the processing circuitry causes the display to display the activity position data and the information about the positioning of the device so as to be superimposed on the blood vessel image.

3. The medical image processing apparatus according to claim 2, wherein, on the basis of the result of the registration, the processing circuitry is configured to calculate a shortest distance between one of the plurality of activity regions and the blood vessel, as the positional relationship.

4. The medical image processing apparatus according to claim 2, wherein
the processing circuitry is configured to calculate a center-of-gravity position of activity amounts in the plurality of activity regions, on a basis of an activity amount of each of a plurality of voxels in each of the plurality of activity regions, and
the processing circuitry is configured to calculate a shortest distance between the center-of-gravity position and the blood vessel as the positional relationship, on the basis of the result of the registration.

5. The medical image processing apparatus according to claim 3, wherein the processing circuitry causes the display to display, over the blood vessel rendered in the blood vessel image, the positioning of the device among the information about the positioning of the device, by using mutually-different hues in accordance with distances from a position related to the shortest distance.

6. The medical image processing apparatus according to claim 5, wherein
the processing circuitry is configured
to calculate a dangerous position having a danger in relation to the positioning of the device, on a basis of structure information of the blood vessel, and
to cause the display to display, over the blood vessel, the dangerous position by using a hue different from the hues.

7. The medical image processing apparatus according to claim 1, wherein
the processing circuitry is configured
to obtain a fluoroscopic image related to the examined subject, and
to cause the display to display the information about the positioning of the device, together with the fluoroscopic image.

8. The medical image processing apparatus according to claim 7, wherein
the processing circuitry is configured
to further obtain geometric information of an imaging system related to imaging the examined subject; and
to calculate, on a basis of the geometric information related to the fluoroscopic image, a target position indicating a target for positioning the device among the information about the positioning of the device, pursuant to a change in the geometric information of the imaging system, and
to cause the display to display the target position so as to be superimposed on the fluoroscopic image.

9. The medical image processing apparatus according to claim 1, wherein
information about the device includes a target position indicating a target for positioning the device in relation to the receiving of the electrical signal, and
as being triggered by the device reaching the target position, the processing circuitry is configured to cause the display to notify that the device has reached the target position.

10. The medical image processing apparatus according to claim 1, wherein the device is a stent having an electrode configured to receive the electrical signal occurring in the activity regions.

11. An X-ray diagnosis apparatus comprising:
an X-ray tube configured to emit X-rays onto an examined subject;
processing circuitry configured
to generate a blood vessel image rendering a three-dimensional blood vessel on a basis of the emission of the X-rays onto the examined subject,
to obtain activity position data related to positions of a plurality of activity regions that bring a site of the examined subject into an activity and the three-dimensional blood vessel image related to the plurality of activity regions,
to perform a registration between the activity position data and the blood vessel image, and
to calculate a positional relationship between at least one of the plurality of activity regions and the blood vessel, on a basis of a result of the registration; and
to cause a display to display, on a basis of the positional relationship, information about positioning a device capable of receiving, within the blood vessel of the examined subject, electrical signals from the activity regions.

12. The X-ray diagnosis apparatus according to claim 11, wherein
the processing circuitry is configured to obtain a fluoroscopic image generated on a basis of fluoroscopic imaging performed on the examined subject and geometric information of an imaging system related to imaging the examined subject,
the processing circuitry is configured
to calculate, on a basis of the geometric information related to the fluoroscopic image, a target position indicating a target for positioning the device among the information about the positioning of the device, pursuant to a change in the geometric information of the imaging system, and
to cause the display to display the target position so as to be superimposed on the fluoroscopic image.

13. A medical image processing method comprising:
obtaining activity position data related to positions of a plurality of activity regions that bring a site of an examined subject into an activity and a blood vessel image rendering a three-dimensional blood vessel related to the plurality of activity regions;
performing a registration between the activity position data and the blood vessel image;
calculating a positional relationship between at least one of the plurality of activity regions and the blood vessel, on a basis of a result of the registration; and
displaying, on a basis of the positional relationship, information about positioning a device capable of receiving, within the blood vessel of the examined subject, electrical signals from the activity regions.

14. The medical image processing method according to claim 13, further comprising:
obtaining a fluoroscopic image generated on a basis of fluoroscopic imaging performed on the examined subject and geometric information of an imaging system related to imaging the examined subject;
calculating, on a basis of the geometric information related to the fluoroscopic image, a target position indicating a target for positioning the device among the information about the positioning of the device, pursuant to a change in the geometric information of the imaging system; and
displaying the target position so as to be superimposed on the fluoroscopic image.
